(19) United States
(12) Patent Application Publication
(10) Pub. No.: US 2007/0053965 A1
(43) Pub. Date: Mar. 8, 2007

(54) Kit comprising a plaster, optionally containing an active substance, and an agent that at least partially reduces skin irritation

(75) Inventors: Heinrich Kugelmann, Aachen (DE); Johannes Bartholomaeus, Aachen (DE); Rasoul Sedaghat Kerdar, Nuernberg (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/509,613

(22) Filed: Aug. 25, 2006

Related U.S. Application Data
(63) Continuation of application No. PCT/EP05/02033, filed on Feb. 25, 2005.

(51) Int. Cl. A61K 9/70; A61F 13/00

(52) U.S. Cl. 424/449; 602/1

(57) ABSTRACT

A kit comprising a plaster, which contains a pharmaceutical active ingredient and is designed to administer the active ingredient transdermally to a human or animal to which the plaster is applied, and an agent that is separate from the plaster and at least reduces skin irritation from the plaster.

### CROSS REFERENCE TO RELATED APPLICATIONS

This application is a continuation of international patent application no. PCT/EP2005/002033, filed Feb. 25, 2005 designating the United States of America, and published in German on Sep. 9, 2005 as WO 2005/082336, the entire disclosure of which is incorporated herein by reference. Priority is claimed based on Federal Republic of Germany patent application no. DE 10 2004 009 904.9, filed Feb. 26, 2004.

### BACKGROUND OF THE INVENTION

The present invention relates to a kit consisting of an active substance plaster for transdermal release of a pharmaceutically active substance and a separate agent that at least partially reduces skin irritations.

It is known that intense contact between plasters and the skin, particularly if it is dry or already damaged, can lead to erythema, pustules, pruritus, or other manifestations of skin irritation. These dermal reactions can occur in up to 30% of the users of plasters to different extents. The consequence is an only moderate acceptance of these products, which can lead to premature termination of therapy.

Therefore the problem set was to overcome this drawback of the prior art to the greatest possible extent and thus to create better acceptance for the use of plasters.

### SUMMARY OF THE INVENTION

This problem has been solved by the provision of a kit consisting of an active substance plaster for transdermal administration of a pharmaceutically active substance and of a separate agent that at least partially reduces skin irritations.

The agent that at least partially reduces skin irritation, preferably a skin friendly agent, is separately packaged and is in a form capable of application to the skin. The advantage of this is that the user can avoid skin irritations by applying the agent immediately before applying the plaster and, moreover, before such irritation is even induced, without having to wait for skin irritation to be reduced by the diffusion of agents from the layers of the plaster, if at all.

Furthermore, the user of the plaster is also able to apply the agent repeatedly if necessary during the plaster therapy, without having to abort the therapy.

The agent provided in the kit can be packaged in individual portions as required for use or can be applied portionwise from a suitable container onto the skin.

Each portion of the agent is preferably such that the adhesive effect of the plaster is not, or only minimally, affected. Preferably the agent is applied to the skin using an applicator. The amount of agent is preferably determined individually according to the size of the plaster.

The agent that at least partially reduces skin irritations can be bottled in a container, such as, for example, a pressure container, a blister package, an ampoule, a bag, a bottle, a capsule, a crucible, a jar, or a tube, from which it is applied to the human or animal skin.

To simplify the application of the agent, an applicator can be provided with the container, said applicator being a removable part of the container, or packaged separately from the container. Suitable for use as an applicator are preferably optionally aseptic spatulas, cotton wool tips, a drip pipette, a brush, a nozzle with a dosage valve, or a roll-on applicator system.

According to the invention, the container used is preferably a pressure container that is equipped with a dispenser and contains a known, preferably environmentally friendly atomizing agent.

The agent that at least partially reduces skin irritations can be present in a solid, liquid or semi-solid form, for example as emulsion, micro-emulsion, cream, ointment, paste, lotion, gel, compressed powder, loose powder, or a stick.

Preferably the agent that at least partially reduces skin irritations is present in liquid or semi-solid form. Gels and creams as semi-solid forms and lotions as liquid forms are preferred. Here the agent, liquid or semi-solid, can be present in a single (aqueous or oily) phase or in an aqueous or oily suspension and/or an emulsion. For example, it can be present in a W/O, W/O/W, or O/W emulsion, in which the oily phase can consist of silicones and/or silicone derivatives.

The agent that at least partially reduces skin irritations preferably contains a compound or substance which is selected from the group consisting of paraffins, silanols, silicones, silicone derivatives, monohydric, dihydric or polyhydric alcohols, natural or synthetic lipids, natural or synthetic waxes, natural or synthetic fats, fatty acids and/or fatty alcohols, natural or synthetic oils, natural or synthetic polymers, starches, proteins, vitamins, compounds with antiphlogogenic or antiphlogistic characteristics, compounds for growth prevention of phlogogenic microorganisms, compounds with anesthetizing characteristics, compounds that are effective as radical scavengers, enzymes, herbal extracts, preservatives, and mixtures thereof consisting of at least two compounds of one class or at least two compounds of different classes. If glycerin is used as a skin friendly, in particular skin smoothing, compound, this is used only in combination with at least one further compound from a different class, preferably at least one compound selected from the preferably used compounds that at least partially reduce skin irritations as mentioned below.

As used herein, the designation “compounds that at least partially reduce skin irritations” includes combinations of the compounds stated as having the desired effect according to the invention.

Suitable silicone derivatives are preferably substituted or unsubstituted polysiloxanes optionally in admixture with acrylate polymers.

Suitable compounds for having antiphlogogenic or antiphlogistic characteristics are preferably allantoin, dexpanthenol, bisabolol, chamazulene, aescin, basic aluminum acetate/tartrate, zinc oxide, tannin, melatonin, balsam of Peru, bismuth gallate, derivatives and/or salts thereof, corticoids, such as, preferably, hydrocortisone, betamethason, fluocinolone acetonide, fluocinonide, prednisolone, methyl prednisolone, triamcinolone, flumetasone, clobetasol, fluprednides, alclometasone, prednicarbate, mometasone, fluticasone, halcinonid, clocortolone, diflucortolone, desoximetasone and/or derivatives thereof, and also antihistamines, such as, preferably, diphenhydramine, dimetindene, isoprenalin, clemastin, bamipine, and derivatives and/or salts thereof.

Compounds for growth prevention of antiphlogogenic microorganisms are preferably benzalkonium chlorides such as, for example, benzethonium chloride, methylhydroxybenzoate, propylhydroxybenzoate, chlorohexidine, dequalinium chloride, clioquinol, sorbic acid, derivatives and/or salts thereof, antiseptics such as, preferably, povidon iodine, iodoform, thymol, tyrothricin, chlorocresol, salicylic acid, ethacridin or polidocanol, derivatives and/or salts thereof and antiphlogogenic agents such as, preferably, framycetin, neomycin, gentamicin, nystatin, erythromycin, tetracyclin, chlorotetracycline, oxytetracyclin, fusidic acid, metronidazole, bacitracin zinc, miconazole, amphotericin B, derivatives and/or salts thereof.

Compounds with anesthetizing characteristics are preferably benzocaine, lidocaine, tetracaine, prilocaine, mepivacaine, and derivatives and/or salts thereof.

Suitable vitamins are preferably Vitamin A derivatives, preferably retinol acetate or retinol palmitate, Vitamin B derivatives, Vitamin C derivatives, such as, for example, the respective palmitates, Vitamin D derivatives, preferably colecaliciferol or Vitamin E derivatives, preferably α-tocopherol acetate.

Suitable enzymes are preferably superoxide dismutases or catalases.

Suitable herbal extracts are preferably extracts of plants such as for example aloe vera, arnica, basil, wild plum (Lat.: Prunus spinosa), greater burdock (Lat.: Arctium lappa), pot marigold (Lat.: Calendula officinalis), camellia (Lat.: Camellia oleifera), clary (Lat.: Salvia clarea), German chamomile (Lat.: Matricaria chamomilla), comfrey (Lat.: Symphytum officinale), echinacea (Lat.: Echinacea angustifolia), cucumber (Lat.: Cucumis Sativus), euphrasia (Lat.: Euphrasia officinalis), ginseng, green tea, lavender, chamomile (Lat.: Chamomilla recutita and Matricaria chamomilla), peppermint (Lat.: Mentha piperita), mugwort, nutmeg, avena (Lat.: Avena sativa), sandal wood, safflower (Lat.: Carthamus tinctorius), soy, melaleuca (Lat.: Melaleuca alternifolia), vetiver grass (Lat.: Vetiveria zizanioides), violet, licorice (Lat.: Glycyrrhiza glabra) and/or witch hazel (Lat.: Hamamelis).

Of the aforementioned compounds preferably those are suitable that also have a skin smoothing action. Suitable as a compound for at least partially reducing skin irritations is at least one skin smoothing compound or substance selected from the group consisting of glycerin, chitosane, hydroxypropylmethylcellulose, oetearyl octanoate, vitamin E, coconut fat, arachis oil, soybean oil, and Butyrospermum parkii (shea butter), whereas hydrophilic compounds such as glycerin are only used in combination with one of the above mentioned skin soothing compounds.

Particularly preferred and suitable is at least one skin friendly compound selected from the group consisting of polymeric compounds, preferably fluorinated polyethers, more preferably polyperfluoromethylisopropyl ether, or silicone derivatives; compounds with antiphlogogenic or antiphlogistic characteristics, preferably corticoids or antihistamines; compounds for prevention of growth of phlogogenic microorganisms, preferably antiseptics or anti-infective agents; and compounds effective as free-radical scavengers, preferably N-acyl ethanolamine.

The aqueous phase of the skin-soothing agent can contain a viscosity increasing agent, selected from the group consisting of cellulose, cellulose-derivatives such as methylcellulose, hydroxymethylcellulose, hydroxyethylcellulose, hydroxypropylcellulose, hydroxypropylmethyl-cellulose, microcrystalline cellulose containing 11% by weight of carboxymethylcellulose sodium (Avicel® RC 591), and carboxymethylcellulose sodium (Blanose®, CMC-Na C300P®, Frimulsion BLC-5®, Tylose C300 P®), polyacrylic acid (Carbopol® 980 NF, Carbopol® 981), locus bean gum (Cesagum® LA-200, Cesagum® LID/150, Cesagum® LN-1), pectins, preferably from citrus fruits or apples (Cesapectin® HM Medium Rapid Set), waxy corn starch (C*Gel 04201®), sodium alginate (Frimulsion ALG (E401)®) guar gum (Frimulsion BM®, Polygum 26/1-75®), iota carrageen (Frimulsion D021®), karaya gum, gellan gum (Kelcogel F®, Kelcogel LT100®), galactomannan (Meyprogat 150®), tara stone flour (Polygum 43/1®), propylene glycol alginate (Protanal-Ester SD-LB®), sodium hyaluronate, tragacanth, tara gum (Vidogum SP 200®), fermented polysaccharide welan gum (K1A96) and xanthane such as xanthane gum (Xantural 180®). The designations in parentheses are examples of trade names under which the respective agents are commercially available.

The agent at least reducing skin irritation may contain an oil concentrating agent such as hydrophobic silicon oxide (for example Aerosil®).

The agent that at least partially reduces skin irritations may contain an emulsifier. Suitable as emulsifiers are anionic surfactants such as sodium dodecylsulfate, sodium cetylstearylsulfate and sodium dioctylsulfosuccinate, amphoteric surfactants such as lecithin, or non-anionic surfactants, for example fatty alcohols and styrenes such as cetyl alcohol, stearyl alcohol, cetylstearyl alcohol and cholestyrene, sorbitan fatty acid ester such as sorbitan monooleate, sorbitan monopalmitate, sorbitan monostearate, sorbitan trioleate, sorbitan tristearate and sorbitan monolaurate, polyoxyethylene sorbitan fatty acid ester such as polyoxyethylene (20) monolaurate, polyoxyethylene (20) monopalmitate polyoxyethylene (20) monostearate and polyoxyethylene (20) monooleate, polyoxyethylene fatty acid glycerides such as macrogol (1500) glyceryl triricinoleate, macrogol glyceryl hydroxystearate, macrogol (1000) glyceryl monolaurate, macrogol (1000) glyceryl monostearate and macrogol (1000) glyceryl monooleate, polyoxyethylene fatty acid esters such as macrogol stearate 400, polyoxyl 40 stearates and polyoxyl 50 stearates, polyethylene fatty alcohol ethers such as polyoxyl 23 lauryl ether, polyoxyl 20 certostearyl ether and polyoxyl 10 polyethers, glycerol fatty acid ester such as glycerol monostearate and macromolecular surfactants such as poloxamer. Silicone emulsifiers are particularly suitable as emulsifiers.

The agent that at least partially reduces skin irritations can contain a skin moisturizing agent as a component, selected, for example, from the group consisting of glycerin, chitosan, fucogel, propylene glycol, dipropylene glycol, butylenes glycol, mannitol, lactic acid, sodium pyrolidoncarboxylic acid, hyaluron acid, salts of the mentioned acids as well as glycine, urea and salts of metals of the first and second main group. Particularly preferred are glycerin, lactic acid, butylenes glycol, urea, and hyaluron acid.

The agent that at least partially reduces skin irritations may contain a preservative. All conventional preservatives are suitable. Among others, they can be preservatives from the group consisting of alcohols (for example chlorobutanol, phenylethyl alcohol or benzyl alcohol), acids (for example sorbic acid, benzoic acid, or boric acid), PHB esters (for example parabenes), phenol derivatives (for example phenol, cresol, or chlorine cresol), quaternary compounds or quaternary ammonium compounds (quats) (for example benzalconium chloride), organic Hg compounds (for example thiomersal, phenyl mercurinitrate or phenyl mercuriborate) and guanines (for example chlorohexidine or chlorohexidine acetate). Mixtures of at least two preservatives can be used, if desired.

The agent that at least partially reduces skin irritations may also contain emulsifiers, stabilizers, anti-oxidants, bactericides, perfumes, antifoam compounds, coloring, pigments, foam stabilizers, and/or electrolytes.

The active substance plaster according to the invention may be constructed according to the reservoir or matrix system (Bauer K. H., Frömming K. H., Führer C, Pharmazeutische Technologie (Pharmaceutical Technology), Pages 381-383; Müller R. H., Hildebrand G. E., Pharmazeutische Technologie: Moderne Arzneiformen (Pharmaceutical Technology: Modern Pharmaceutical Forms), Chapter 8.

In the matrix-system the plaster containing the active substance can preferably consist of a base, a layer containing the active substance, an adhesive layer, and a removable protective film. The layer containing the active substance can at the same time be the adhesive layer, in which the active substance is present in a matrix in dissolved and/or dispersed form together with the adhesive.

Preferably, the adhesive layer and the layer containing the active substance are separate layers, whilst the adhesive may be applied to the layer containing the active substance over the entire area thereof or only a portion thereof or only a circular portion thereof.

If the plaster according to the invention is constructed according to the reservoir system, the adhesive layer may be applied to the entire area of, or as a ring surrounding, the reservoir membrane of the reservoir system.

If the plaster according to the invention does not contain a separate layer containing an active substance, the adhesive layer may be applied to the base entirely or partially, for example in segments, with or without areas containing active substances.

Preferably, pressure-sensitive adhesives are used for the adhesive layer of the plaster. Examples of suitable adhesives are polymers such as polyacrylates, polyvinyl ethers, polyisobutylene (PIB), styrene/isoprene copolymers or butadiene/styrene copolymers or polyisoprene rubber. Other suitable adhesives are silicone adhesives such as, for example, optionally cross-linked polydimethylsiloxanes. Resins such as, for example, esters of glycinen, glycerin, or pentaerythrol, or hydrocarbon resins such as polyterpene are also suitable. Acrylate-based adhesives are produced by polymerization of acrylates, methacrylates, alkyl acrylates and/or alkyl methacrylates, optionally with a further α,β-unsaturated monomer such as acrylamide, dimethylacrylamide, dimethylaminoethyl acrylate, hydroxyethyl acrylate, hydroxypropyl acrylate, methoxyethyl acrylate, methoxyethyl methacrylate, acrylonitrile, and/or vinyl acetate.

The adhesive layer may also contain additional adjuvants, such as plasticizers, for example phthalates such as dibutyl phthalates, mineral oils, esters of citric acid, or esters of glycerin, skin penetration enhancers, fillers (such as zinc oxide or silica), cross linkers, preservatives, and/or lipophilic solvents.

The base, or top layer, of the plaster is preferably impermeable and inert to the substances present in the layer containing the active substance and to the substances in the adhesive layer, particularly to the active substance which may be present therein, and can consist of polyesters, for example polyethylene phthalates, polyolefins, such as polyethylenes, polypropylenes, or polybutylenes, polycarbonates, polyethylene oxides, polyterephthalates such as polyethylene terephthalates, polyurethanes, polystyrenes, polyamides, polyimides, polyvinyl acetates, polyvinyl chlorides, and/or polyvinylidene chlorides, copolymers such as acrylonitrile/butadiene/styrene copolymer containing fibers, textile fibers, and/or mixtures thereof, which may be metallized or pigmented if necessary. The base may also consist of a combination of a metal foil and a polymeric layer.

The plaster according to the invention is suitable for transdermal administration of any systemic, ie transdermally effective, pharmaceutically active substance. Preferably the plaster according to the invention is suitable for transdermal (systemic) release of at least one pharmaceutically active substance from the group consisting of analgetics, local anesthetics, hormones, contraceptives, vaccines, immune modulators, anti-allergic agents, antihistamines, cardiac agents, antihypertonics, psychotropics, anti-rheumatic agents, and enzymes. It is particularly suitable for transdermal administration of opioids, such as, for example, buprenorphine, tentanyl, or morphine.

The required dosage for transdermal administration of the active substance is known to the person skilled in the art and is dependent on the duration of the therapy, among other factors.

A matrix layer of the active substance plaster according to the invention can, along with the preferred pharmaceutical agent, contain matrix forming polymers, plasticizers, permeation enhancers, lipophilic solvents, cross linking agents, preservatives, emulsifiers, thickening agents, and/or conventional reservoir membrane system additives, or reservoir transdermal system additives together with the adhesive, if present.

Matrix forming polymers can usually be film forming polymers such as, for example, hydroxypropylcellulose, carboxymethylcellulose, polyethylene s, chlorinated polyethylenes, polypropylene s, polyurethanes, polycarbonates, polyacrylic acid esters, polyacrylates, polymethacrylates, polyvinyl alcohols, polyvinyl chlorides, polyvinylidene chlorides, polyvinylpyrrolidones, polyethylene therephthalates, polytetrafluoroethylenes, ethylene/propylene copolymers, ethylene/ethyl acrylate copolymers, ethylene/vinyl acetate copolymers, ethylene/vinyl alcohol copolymers, ethylene/vinyloxyethanol copolymers, vinyl chloride/vinyl acetate copolymers, vinylpyrrolidone/ethylene/vinyl acetate copolymers, rubbers, rubber-like synthetic homopolymers, copolymers or block polymers, silicones, silicone-derivatives such as polysiloxane/polymethacrylate copolymers, cellulose derivatives such as ethyl cellulose or cellulose ether, and/or mixtures thereof. If the layer containing the active substance is at the same time the adhesive layer, it preferably contains at least one of the above mentioned adhesives along with at least one of the listed polymers.

Suitable lipophilic solvents are N-methyl-2-pyrrolidone, lauryl pyrrolidone, triethanolamine, triacetin, diethylene glycol monoethyl ether, and derivatives of fatty acids or fatty alcohols.

If the active substance plaster is constructed according to the reservoir system, the reservoir membrane can consist of inert polymers such as, for example, polyethylenes, polypropylenes, polyvinyl acetates, polyamides, ethylene/vinyl acetate copolymers, and/or silicones. The reservoir membrane allows for a controlled release of the active substance.

The matrix or the reservoir containing the active substance can also contain a solvent, such as, for example, water, ethanol, 1-propanol, isopropanol, a low molecular weight, polyhydric alcohol, for example propylene glycol or glycerin, or an ester, such as isopropyl myristate, or mixtures thereof.

Preservatives for the matrix or reservoir containing the active substance can be antioxidants, such as vitamin E, butylhydroxytoluene, butylhydroxyanisol, ascorbic acid, ascorbyl palmitate, and/or chelating agents, such as disodium methylenediamine tetraacetic acid, potassium nitrate, or sodium nitrate.

The matrix or reservoir containing the active substance can also contain known and conventional permeation enhancers.

The plaster can also contain viscosity enhancing agents in the matrix or reservoir containing the active substance such as, for example, cellulose derivatives or natural or synthetic rubbers. The pharmaceutical agent in the plaster according to the invention is preferably present in a matrix layer.

The release of the agent is preferably controlled.

The plaster of the kit according to the invention can also contain, in one or more layers, at least one plasticizer selected from the group consisting of long-chain alcohols such as dodecanol, undecanol, octanol, esters of carboxylic acid with polyethoxylated alcohols, diesters of aliphatic dicarboxylic acids such as adipic acid, and medium-chain triglycerides of caprylic acid and/or capric acid, coconut oil, polyhydric alcohols such as for example propane-1,2-diol, esters of polyhydric alcohols such as glycerin with levulic acid or caprylic acid, and etherified polyhydric alcohols.

The protective film may be composed of polyethylene, polyesters, polyethylene terephthalate, polypropylene, polysiloxane, polyvinyl chloride, or polyurethane or optionally of treated paper fibers, such as, for example, cellophane, and optionally contain a layer of silicone, fluorosilicone, or fluorocarbon.

The production of the plasters is carried out by known manufacturing techniques for plasters comprising process steps such as laminating, die cutting, delaminating, uncoiling, cutting, recoiling, mounting or metering (see Verpackungs-Rundschau 4/2002, 83-84).

The plaster of the invention can be applied to humans or animals, and skin irritations that are caused due to usage of plasters are largely avoided.

When using the kit of the invention, the skin area should be cleaned (washed and dried) before application, if possible. Subsequently the skin friendly agent that at least partially reduces skin irritations should be applied thinly, ie, as a layer of <5 μm, preferably between 0.5 μm and 2 μm. After application of the skin friendly agent that at least partially reduces skin irritations, the plaster is applied to the skin with slight pressure.

### EXAMPLES

### Example 1

In order to test a kit according to the invention in comparison with the application of only a plaster the following test was carried out:

Five test subject persons each applied a 2×2 cm piece of matrix plaster that contained 0.5% by weight of Capsaicin to a hair-free area of the forearm, in one test after this area had been sprayed with a solution of a mixture of hexamethylene disiloxane and acrylate polymer in ethanol (solids content ≈2% by weight, trade name Cavilon® of 3M Health care, MN, USA) and after a film had formed having a thickness of ≈1 μm following evaporation of volatile fractions, and in another test without previous application to the skin of an agent for preventing skin irritations.

After three days the plasters were removed by the test subjects and any skin irritations were evaluated according to the following assessment scale immediately after removal and also after a further 24 hours. The test results are listed in the following Table 1.

TABLE 1

| | |
|---|---|
| Plaster | Plaster |
| without the use of | with the use of |

a film of hexamethylene disiloxane and acrylate
polymer

| Person | after 3 d | after 4 d | after 3 d | after 4 d |
|---|---|---|---|---|
| 1 | +++ | +++ | (+) | − |
| 2 | ++ | +++ | − | − |
| 3 | +++ | ++ | (+) | − |
| 4 | ++++ | +++ | (+) | − |
| 5 | ++ | +++ | − | − |

− = no skin irritation
(+) = scarcely visible skin irritation
+ = weak skin irritation
++ = spots of strong skin irritation
+++ = strong irritation
++++ = strong irritation with pustules

The test results confirm that the application of a compound for preventing skin irritation to the skin prior to usage of a plaster effectively prevents irritations to a large extent.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A kit comprising:
an active substance plaster for transdermal release of at least one pharmaceutically active substance selected from the group consisting of opioids, local anesthetics, hormones, contraceptives, vaccines, immunomodulators, anti-allergic agents, antihistaminic agents, cardiac agents, antihypertensive agents, psychotropic agents, antirheumatic agents and enzymes, and
a separate agent for at least partially reducing skin irritation.

2. A kit according to claim 1, wherein said agent for reducing skin irritation is present in a form capable of being applied directly to the skin.

3. A kit according to claim 1, wherein said agent for reducing skin irritation is present in individually packed portions.

4. A kit according to claim 1, wherein said agent for reducing skin irritation is present in a container.

5. A kit according to claim 4, wherein said container comprises an applicator for said agent.

6. A kit according to claim 4, wherein said container is a pressure vessel, a blister package, an ampoule, a bag, a flask, a tin, a crucible, a jar, or a tube.

7. A kit according to claim 1, wherein said agent for reducing skin irritation is present in semisolid form.

8. A kit according to claim 7, wherein said agent for reducing skin irritation is present in the form of a gel or cream.

9. A kit according to claim 1, wherein said agent for reducing skin irritation is present in liquid form.

10. A kit according to claim 9, wherein said agent for reducing skin irritation is present in form of a lotion.

11. A kit according to claim 1, wherein the adhesive action of the plaster is substantially undiminished by said agent for reducing skin irritation.

12. A kit according to claim 1, wherein said agent for reducing skin irritation comprises at least one compound selected from the group consisting of paraffins; silanols; silicones; silicone derivatives; monohydric, dihydric, or polyhydric alcohols; natural and synthetic lipids; natural and synthetic waxes; natural and synthetic fats, fatty acids and fatty alcohols; natural and synthetic oils; natural and synthetic polymers; starches; proteins; vitamins; compounds with antiphlogogenic or antiphlogistic properties; compounds for preventing growth of phlogogenic microorganisms; compounds with anesthetic properties; compounds effective as radical interceptors; enzymes; vegetable extracts; preservatives, and mixtures thereof comprising at least two compounds of one class or at least two compounds of different classes.

13. A kit according to claim 1, wherein said agent comprises at least one compound selected from the group consisting of fluorinated polyethers, silicon derivatives, corticoids, antihistamines, antiseptic agents, anti-infective agents, and N-acyl ethanolamine.

14. A kit according to claim 1, wherein said pharmaceutically active substance is an opioid selected from the group consisting of buprenorphine, fentanyl, and morphine.
